(19) United States
(12) Patent Application Publication
(10) Pub. No.: US 2009/0048436 A1
(43) Pub. Date: Feb. 19, 2009

(54) Methods of synthesizing chemically cleavable phosphoramidite linkers

(76) Inventors: Keith Anderson, Mass Beach, CA (US); Michael Jensen, Los Gatos, CA (US); Ronald W. Davis, Palo Alto, CA (US); Charles K. Brush, Whitefish Bay, WI (US); Kaizhang He, Whitefish Bay, WI (US)

Correspondence Address: THOMPSON HINE L.L.P.;Intellectual Property Group, P.O. BOX 8801, DAYTON, OH, 45401-8801, US

(21) Appl. No.: 11/893,614

(22) Filed: Aug. 15, 2007

(51) Int. Cl. C07H 21/00

(52) U.S. Cl. 536/25.3

(57) ABSTRACT

The present invention provides a method of synthesizing phosphoramidite linkers that are useful for the production of synthesizing two or more oligonucleotides in tandem. The inventive linker has the following desirable properties: (i) enhanced stability to alkali conditions versus the linkers previously published, (ii) cleaves to produce 5′ and 3′ ends that are fully biologically compatible, (iii) cleaves completely under conditions that are already used in cleavage/deprotection processes so it is fully compatible with conditions that are common in laboratories and does not require additives that necessitate further purification after cleavage, (iv) integrates easily onto commercially available synthesizers because it is compatible with standard coupling chemistry, and (v) is compatible with DNA, RNA, forward, reverse, and LNA, synthesis chemistries. In addition, the inventive linkers may be coupled to a solid support. Thus, the inventive linkers provide a significant advancement in the state of the art.

This invention was made in part with government support under grant no. HG00205 awarded by the National Institutes of Health (NIH). The government has certain rights in this invention.

### FIELD OF THE INVENTION

The present invention relates generally to biochemistry. More particularly, the present invention relates to methods of synthesizing chemically cleavable phosphoramidite linkers.

### BACKGROUND

Tandem oligonucleotide synthesis involves stepwise synthesis of two or more oligonucleotides end-to-end in a tandem manner on the surface of a solid-phase support. Cleavage and deprotection of the linked oligos results in two (or more) different oligos in the deprotection solution. Tandem oligonucleotide synthesis is considered especially advantageous for polymerase chain reaction (PCR), in which two oligonucleotides must perform a reaction in the same reaction vessel.

Several methods have been proposed to accomplish tandem oligonucleotide synthesis, but they all have disadvantages. It was first proposed that enzymatic processes could be used to cleave two primers that were synthesized end-to-end by exploiting the specific recognition of Uracil DNA Glycosylase (UDG) enzyme for uridine residues. UDG recognizes uridine, which is not typically present in synthetic DNA, and catalyzes the removal of the uridine nucleobase from the DNA backbone. The DNA backbone at the abasic site is highly susceptible to breakage if the DNA is heated to about 90° C., if it is treated by Human Apurinic Exonuclease (APE), or if it is treated chemically with N,N′-dimethylethylenediamine (DMED). Unfortunately, it was found that UDG and APE preferred double stranded substrates and thus did not reliably create abasic sites or break the DNA backbone of single-stranded DNA oligonucleotides (ssDNA), and thus these treatments proved unsuccessful in breaking a single stranded oligonucleotide. DMED was very effective in breaking the abasic site's backbone generated by UDG, however it too proved to be less than ideal for producing two PCR primers from a synthetic oligonucleotide because it left a 3′ phosphate or a 3′ ring-opened sugar on the 5′ end of the cut site. Because polymerases and most other DNA acting enzymes require a 3′ hydroxyl in order for the DNA to initiate enzymatic activity, DMED could not be used because it would not reliably leave a 3′ hydroxyl.

Commercially available chemical methods were also investigated. One method was based on a modified phosphoramidite where each base was chemically separated from the 3′ phosphate by a chemically cleavable linker. Two structures were proposed for separating the phosphate from the 3′ oxygen on the nucleobase. In one case, the phosphate was separated by a sulfone group, which is highly base-labile and cleaves quickly in ammonium hydroxide, a chemical that is already used to deprotect the nucleobases of an oligonucleotide. There are two problems with this approach. First, the sulfone group is sufficiently base-labile so that the phosphoramidite would quickly degrade before it was chemically coupled (incorporated) into the oligonucleotide. Second, degradation may occur during storage, particularly for oligonucleotides containing more basic nucleosides. Another version of this linker cleaved but left a 5′ ethylphosphate, which is incompatible with several biological processes, which require a 5′ phosphate or a 5′ hydroxyl. Accordingly, there is a need in the art to develop a new method of tandem oligonucleotide synthesis that produces oligonucleotides that are more stable during storage and synthesis and more suitable for downstream reactions.

### SUMMARY OF THE INVENTION

The present invention provides phosphoramidite linkers that are useful for the production of synthesizing two or more oligonucleotides in tandem. The inventive linkers have the following desirable properties: (i) enhanced stability to alkali conditions versus the linkers previously published, (ii) cleaves to produce 5′ and 3′ ends that are fully biologically compatible, (iii) cleaves completely under conditions that are already used in cleavage/deprotection processes so it is fully compatible with conditions that are common in laboratories and does not require additives that necessitate further purification after cleavage, (iv) integrates easily onto commercially available synthesizers because it is compatible with standard coupling chemistry, and (v) is compatible with DNA, RNA, forward, reverse, and LNA synthesis chemistry. In addition, the inventive linkers may be coupled to a solid support. Thus, the inventive linkers provide a significant advancement in the state of the art.

In one embodiment, the present invention provides a compound having formula I:

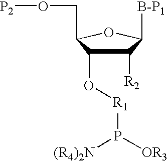

wherein
B is a nucleobase;
P₁ is an acyl, an aroyl, a phenoxyacetyl, an isopropylphenoxyacetyl, a t-butylphenoxyacetyl, an acetyl, a benzoyl, an isobutyryl, a levulinoyl, a dialkylformamidino, an fmoc, or a photolabile protecting group;
P₂ is a dimethoxytrityl, a monomethoxytrityl, a levulinoyl, an ArCO, a silyl, or a photolabile protecting group;
R₁ is a base-labile group;
R₂ is a hydrogen, a fluoro, a protected amino, a protected hydroxyl, an O-alkyl, an O-alkylalkoxy, or a secondary amine;
R₃ is a phosphorus protecting group;
R₄ is an alkyl or (R₄)₂ forms a cyclic secondary amine; and
O, P, and N have their normal meanings of oxygen, phosphorous and nitrogen.

In another embodiment, the present invention provides a material having formula II:

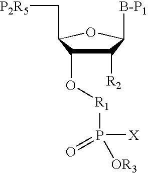

wherein
B is a nucleobase;
P₁ is an acyl, an aroyl, phenoxyacetyl, an isopropylphenoxyacetyl, a t-butylphenoxyacetyl, an acetyl, a benzoyl, an isobutyryl, a levulinoyl, a dialkylformamidino, an fmoc, or a photolabile protecting group;
P₂ is a dimethoxytrityl, a monomethoxytrityl, a levulinoyl, an ArCO, a silyl or a photolabile protecting group;
R₁ is a base-labile group;
R₂ is a hydrogen, a fluoro, a protected amino, a protected hydroxyl, an O-alkyl, an O-alkylalkoxy, or a secondary amine;
R₅ is at least one nucleotide;
R₃ is a phosphorous protecting group;
X is a solid support; and
O, P, and N have their normal meanings of oxygen, phosphorous and nitrogen.

In yet another embodiment, the present invention provides a method of synthesizing the compound having formula I. According to this method, a compound having formula III:

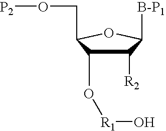

is provided, wherein
B is a nucleobase;
P₁ is an acyl, an aroyl, phenoxyacetyl, an isopropylphenoxyacetyl, a t-butylphenoxyacetyl, an acetyl, a benzoyl, an isobutyryl, a levulinoyl, a dialkylformamidino, an fmoc, or a photolabile protecting group;
P₂ is a dimethoxytrityl, a monomethoxytrityl, a levulinoyl, an ArCO, a silyl or a photolabile protecting group;
R₁ is a base-labile group;
R₂ is a hydrogen, a fluoro, a protected amino, a protected hydroxyl, an O-alkyl, an O-alkylalkoxy, or a secondary amine; and
O and H have their normal meanings of oxygen and hydrogen.

The compound having formula III is reacted with one of two types of compounds. In one aspect of this embodiment, the compound having formula III is reacted with about 1-1.5 equivalents of an O-protected bis-dialkylaminophosphodiamidite, (R′₁O—P—(NR′₂)₂ where R′₂ is a dialkyl or (NR′₂)₂ forms a cyclic secondary amine, and R′₁ is a protecting group. In another aspect of this embodiment, the compound having formula III is reacted with 1-1.5 equivalents of chloro-β-cyanoethyl-N′N′-diisopropylphosphoramidite in the presence of a tertiary amine.

In yet another embodiment, the present invention provides a method of synthesizing at least two oligonucleotides in tandem. According to this method, a first nucleotide is synthesized. The compound having formula I is then incorporated into this first oligonucleotide. Next, a second oligonucleotide is synthesized, where the second oligonucleotide is coupled to the compound having formula I. Finally, the first and second oligonucleotides are cleaved from the compound having formula I.

In a final embodiment, the present invention provides a method of synthesizing an oligonucleotide. According to this method, the material having formula II is provided and a sequence of bases is coupled to this material until the oligonucleotide is synthesized.

### BRIEF DESCRIPTION OF THE FIGURES

The present invention together with its objectives and advantages will be understood by reading the following description in conjunction with the drawings, in which:

FIG. 1 shows an example of synthesis of tandem oligonucleotides according to the present invention.

FIG. 2 shows an example of synthesis of oligonucleotides on a solid support according to the present invention.

FIG. 3 shows an example of synthesis of a phosphoramidite linker according to the present invention.

FIG. 4 shows an example of cleavage of tandem DNA oligonucleotides according to the present invention.

FIG. 5 shows an example of functionality of a cleaved tandem oligonucleotide primer pair in a PCR reaction according to the present invention.

FIG. 6 shows an example of quality of DNA synthesized from a phosphoramidite linker coupled to either a polystyrene or glass support.

FIG. 7 shows an example of synthesis and cleavage of tandem RNA oligonucleotides according to the present invention.

### DETAILED DESCRIPTION OF THE INVENTION

In one embodiment, the present invention provides a compound having formula I (hereafter referred to as the phosphoramidite linker:

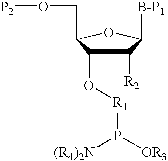

wherein:
B is a nucleobase;
P₁ is an acyl, an aroyl, a phenoxyacetyl, an isopropylphenoxyacetyl, a t-butylphenoxyacetyl, an acetyl, a benzoyl, an isobutyryl, a levulinoyl, a dialkylformamidino, an fmoc, or a photolabile protecting group;
P₂ is a dimethoxytrityl, a monomethoxytrityl, a levulinoyl, an ArCO, a silyl or a photolabile protecting group;
R₁ is a base-labile group;
R₂ is a hydrogen, a fluoro, a protected amino, a protected hydroxyl, an O-alkyl, an O-alkylalkoxy, a secondary amine, or a phosphorous protecting group; and
R₃ is a phosphorus protecting group;
R₄ is an alkyl or (R₄)₂ forms a cyclic secondary amine; and
O, P, and N have their normal meanings of oxygen, phosphorous and nitrogen.

In a preferred aspect of this embodiment, R₁ is

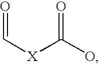

where x is an alkyl, an alkoxyalkyl, an aryl aralkyl, or an ether. In a particularly preferred aspect of this embodiment, R₁ is a succinate, a malonate, a glutarate, an adipate, a diglycolate, a catechol, or an analog or derivative thereof. A key quality of R₁ is that it be a bi-functional group in which both functions are base labile. Preferably, the hydroxyl in R₂ is protected by 2′TBDMS (t-butyldimethylsilyl), 2′TOM (triisopropylsilyloxymethyl), or 2′ACE (bis-acetoxyethylorthoformate). In a particularly preferred aspect of this embodiment, the hydroxyl in R₂ is protected by a silyl group. In another preferred aspect of this embodiment, the photolabile protecting group is 2-(2-nitrophenyl)-propoxycarbonyl, 2-(2-nitrophenyl) propoxycarbonyl piperidine (NPPOC-pip), 2-(2-nitrophenyl)-propoxycarbonyl hydrazine (NPPOC-Hz), or MeNPOC (3,4-methylenedioxy-6-nitro-phenylethyloxycarbonyl). The nucleobase according to this embodiment of the invention may be any type of nucleobase, including but not limited to a deoxyribonucleobase, a ribonucleobase, or analogs or derivatives thereof.

In another embodiment, the present invention provides a material having formula II (hereafter referred to as the phosphoramidite linker material):

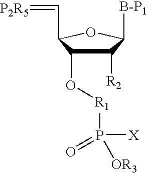

wherein:
B is a nucleobase;
P₁ is an acyl, an aroyl, a phenoxyacetyl, an isopropylphenoxyacetyl, a t-butylphenoxyacetyl, an acetyl, a benzoyl, an isobutyryl, a levulinoyl, a dialkylformamidino, an fmoc, or a photolabile protecting group;
P₂ is a dimethoxytrityl, a monomethoxytrityl, a levulinoyl, an ArCO, a silyl or a photolabile protecting group;
R₁ is a base-labile group;
R₂ is a hydrogen, a fluoro, a protected amino, a protected hydroxyl, an O-alkyl, an O-alkylalkoxy, or a secondary amine;
R₅ is at least one nucleotide;
R₃ is a phosphorous protecting group;
X is a solid support; and
O, P, and N have their normal meanings of oxygen, phosphorous and nitrogen.

In a preferred aspect of this embodiment, R₁ is

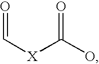

where x is an alkyl, an alkoxyalkyl, an aryl aralkyl, or an ether. In a particularly preferred aspect of this embodiment, R₁ is a succinate, a malonate, a glutarate, an adipate, a diglycolate, a catechol, or an analog or derivative thereof. Preferably, the hydroxyl in R₂ is protected by 2′TBDMS, 2′TOM, or 2′ACE. In a particularly preferred aspect of this embodiment, the hydroxyl in R₂ is protected by a silyl group. In another preferred aspect of this embodiment, the photolabile protecting group is 2-(2-nitrophenyl)-propoxycarbonyl, 2-(2-nitrophenyl) propoxycarbonyl piperidine (NPPOC-pip), 2-(2-nitrophenyl)-propoxycarbonyl hydrazine (NPPOC-Hz), or MeNPOC. The nucleobase according to this embodiment of the invention may be any type of nucleobase, including but not limited to a deoxyribonucleobase, a ribonucleobase, or analogs or derivatives thereof. Also preferably, the solid support is a solid support matrix. The solid support may be, but is not limited to, controlled pore glass, polystyrene, or an oligonucleotide array.

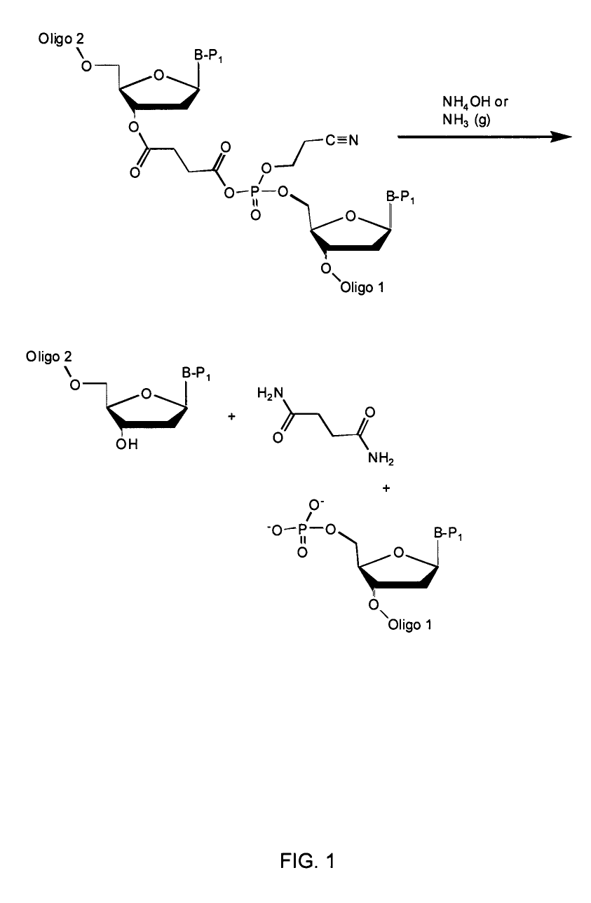

In another embodiment, the present invention provides a method of synthesizing at least two oligonucleotides in tandem. According to this method, a first oligonucleotide is synthesized. Next, the phosphoramidite linker is incorporated into the first oligonucleotide. Next, a second oligonucleotide is synthesized, where the second oligonucleotide is coupled to the phosphoramidite linker. Finally, the first and second oligonucleotides are cleaved from the phosphoramidite linker. Importantly, once the second oligonucleotide is cleaved from the phosphoramidite linker, the 3′ end of the second oligonucleotide ends up as —OH (after deprotection), the succinate linker is lost, and the phosphorous becomes part of the 5′-phosphate of the first oligo, as shown in FIG. 1.

The second oligonucleotide may be coupled to the phosphoramidite linker using any coupling chemistry, including any standard coupling chemistry known in the art. In an exemplary embodiment, the second oligonucleotide is coupled to the phosphoramidite linker using the following method, called the phosphoramidite method. According to this method, coupling reactions are catalyzed by a weakly acidic compound, which protonates the amidite nitrogen; the conjugate base of the compound serves as a nucleophile to activate the phosphorus atom. The electropositive phosphorous subsequently attacks the electronegative oxygen (on the 5′ end of the support-bound nucleoside/oligomer). This chemical attack results in a phosphite triester, which is stabilized by oxidation to the pentavalent phosphate triester during a subsequent step. Activators typically used for this reaction include, but are not limited to, 1-H-Tetrazole, 5-ethylthio-1H-tetrazole (ETT), 5-benzylthio-1H-tetrazole (BTT), dicyanoimidazole (DCI), a pyridinium salt and a trifluomethanesulfonate salt.

The inventive method may further include deprotecting the oligonucleotides, using techniques known in the art. These deprotected oligonucleotides may be used directly in, e.g., PCR reactions, sequencing reactions, or ligation reactions. As such, the oligonucleotides may be, but are not limited to, PCR primers, synthetic genes, DNA oligonucleotides, or RNA oligonucleotides.

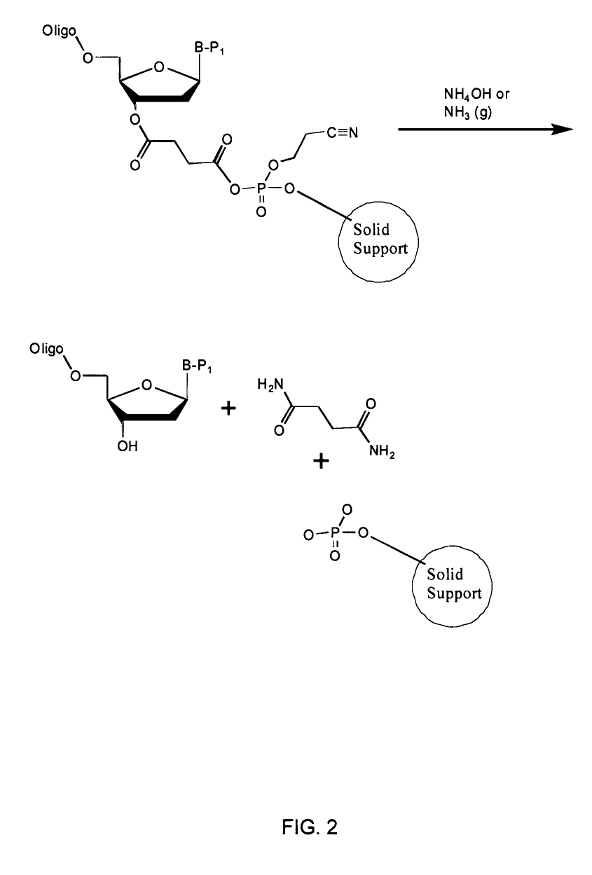

In another embodiment, the present invention provides a method of synthesizing an oligonucleotide, as shown in FIG. 2. This method includes providing a phosphoramidite linker material and coupling a sequence of bases to the material until the oligonucleotide is synthesized. The oligonucleotide may then be cleaved from the phosphoramidite linker material. Any oligonucleotides may by synthesized according to the present invention, including but not limited to PCR primers, synthetic genes, DNA oligonucleotides, or RNA oligonucleotides. The inventive method may further include deprotecting the oligonucleotides, using techniques known in the art. These deprotected oligonucleotides may be used directly in, e.g., PCR reactions, sequencing reactions, or ligation reactions.

The present invention also provides a method of synthesizing the phosphoramidite linker. According to this method, one first provides a compound having formula III:

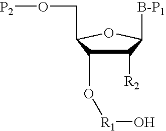

wherein
B is a nucleobase;
P₁ is an acyl, an aroyl, phenoxyacetyl, an isopropylphenoxyacetyl, a t-butylphenoxyacetyl, an acetyl, a benzoyl, an isobutyryl, a levulinoyl, a dialkylformamidino, an fmoc, or a photolabile protecting group;
P₂ is a dimethoxytrityl, a monomethoxytrityl, a levulinoyl, an ArCO, a silyl or a photolabile protecting group;
R₁ is a base-labile group;
R₂ is a hydrogen, a fluoro, a protected amino, a protected hydroxyl, an O-alkyl, an O-alkylalkoxy, or a secondary amine; and
O and H have their normal meanings of oxygen and hydrogen.

In this embodiment, the compound having formula III is then reacted with about 1-1.5 equivalents of an O-protected bis-dialkylaminophosphodiamidite, (R′₁O—P—(NR′₂)₂ wherein R′₂ is a dialkyl or (NR′₂)₂ forms a cyclic secondary amine, and R′₁ is a protecting group. (NR′₂)₂ may be, but is not limited to, piperidine, morpholine, or pyrrolidine. R′₁ may be, but is not limited to, methyl, β-cyanoethyl, allyl, or nitrophenethyl. In a preferred aspect of this embodiment, the O-protected bis-dialkylaminophosphodiamidite is β-cyanoethyl-N,N,N′,N′-tetraisopropylphosphordiamidite. Also preferably, 0.05-1.5 equivalents of an activator is added to the O-protected bis-dialkylaminophosphodiamidite. The activator may be, but is not limited to, 1H-tetrazole, S-ethylthiotetrazole, 5-benzylthio-1H-tetrazole, 4,5-dicyanoimidiazole, a trifluoromethylsulfonic acid salt, or a pyridinium salt.

In another embodiment, the compound having formula III is reacted with about 1-1.5 equivalents of chloro-β-cyanoethyl-N′N′-diisopropylphosphoramidite in the presence of a tertiary amine.

In either embodiment, the reaction is preferably accomplished at room temperature for between about 1 and about 5 hours. Also preferably, dichloromethane is added when the reaction is complete in order to form a solution and facilitate the wash step. The solution is then preferably washed with a 5% aqueous NaHCO₃ solution and a saturated NaCl solution. The organic phase of the solution, which contains the phosphoramidite linker, may then be isolated using standard techniques known in the art. Preferably, the organic phase is isolated a pH in the range of between about 7.5 and about 9.5. Preferably, the organic phase is then dried with Na₂SO₄, filtered, and evaporated until no further solvent is distilled over, using techniques known in the art. Preferably, the organic phase is also evaporated, where the evaporating dries the organic phase Phosphoramidite linker prepared according to the present invention is stable for at least 2.5 years at −40° C and at least two days at ambient temperature.

The inventive method may further include coupling the phosphoramidite linker to a solid support, using chemistries known in the art. The solid support may be, for example, a solid support matrix, a controlled pore glass, a polystyrene, or an oligonucleotide array.

The present invention may be used for numerous applications. The following is a list of exemplary, but non-limiting examples of applications.

One important example is the use of the inventive linker to produce PCR primers. In this way, both primers could be prepared in a single well of a multi-well plate, reducing errors and volumes and cost.

Another important example is synthesis of oligonucleotides on microarrays. Following coupling of the phosphoramidite linker to a microarray using standard phosphoramidite coupling procedures, further coupling of phosphoramidite bases can proceed until full length oligonucleotides are built on the solid support. In the case of a microarray, this will enable the production of tens of thousands of unique oligonucleotides in parallel on the array. As the phosphoramidite linker used to tether the first base on the 3′ end of the oligonucleotide is cleavable, all of the synthesized oligos may then be released into solution and used. An application for the large scale production of thousands of oligonucleotides could be the synthesis of whole genes or genomes from the oligos produced on a single microarray.

The inventive linker could be also used for synthesizing RNA oligonucleotides. An application receiving particular attention recently is the use of RNA duplexes for RNA interference (RNAi) studies. RNA duplexes, when designed properly, have been shown to reduce the expression of target genes in vivo, effectively “knocking down” the level of gene expression. Since the RNA oligonucleotides are synthesized in the single stranded form and then pooled with their compliment to form a duplex, time and money could be saved by synthesizing both strands of an RNA duplex in the same reaction vessel.

Another application includes the construction and assembly of synthetic genes where the sense and anti-sense strands are synthesized in tandem. Upon cleavages of the two strands (lengths dependent upon complimentary melting temperatures (Tm) of overlapping regions of homology), the downstream oligonucleotide will hybridize with the upstream strand, still support-bound, creating a double stranded fragment of DNA. This technique also utilizes the presence of the 5′ phosphate inherent upon complete cleavage and removal of the carboxylic-phosphoric acid mixed anhydride. Furthermore, this saves on the cost of additional phosphorylation reagent ordinarily needed to modify the 5′ region of the upstream strand.

### EXAMPLES

Synthesis of the Phosphoramidite Linker

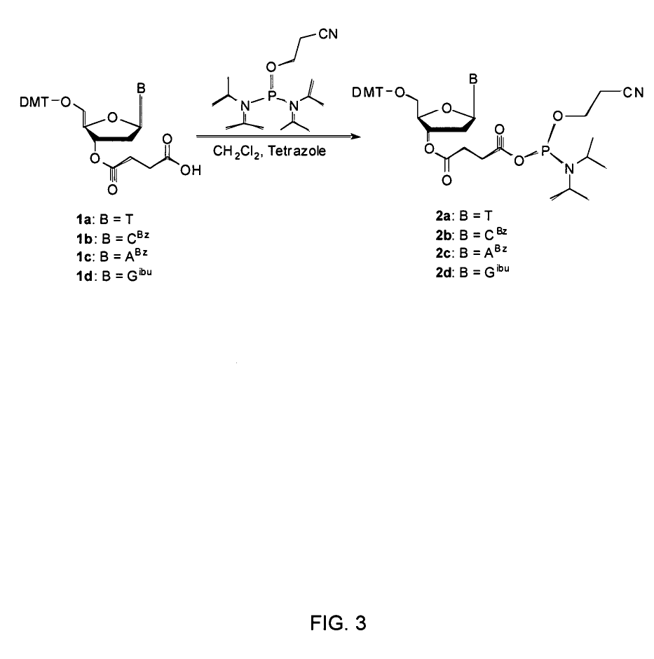

In this example, shown in FIG. 3, succinate phosphoramidite linkers were synthesized. A similar procedure would be used for other phosphoramidite linkers. Nucleoside-3′-O-succinate (1a-1d, Thermo Fisher Scientific (Milwaukee)) was dissolved in dichloromethane in a flask under an argon atmosphere. β-Cyanoethyl-N,N,N′N′-tetraisopropylphosphordiamidite (1 eqv.) was added followed by 1H-tetrazole (1.2 eqv.). The reaction was stirred at room temperature for 1-5 hrs. When complete, dichloromethane was added to the reaction mixture and the resulting solution was washed with 5% aq. NaHCO₃ and saturated NaCl solutions. The organic phase was dried (Na₂SO₄), filtered, and evaporated to dryness. The phosphoramidite (2a-2d) was obtained as white foam with 91-97% HPLC purity. The succinate phosphoramidite linkers (2a-2d) were very stable under common storage conditions.

To determine the stability of succinate phosphoramidite linkers, the products were stored at −40° C. under argon. The linker was then left at room temperature for one hour prior to analysis. Product purity was tested by HPLC and, in some cases, ³¹P NMR (CDCl₃). Results for Bz-dC succinate amidite are shown in Table 1.

TABLE I

| Date | Timepoint | HPLC (%) | ³¹P NMR* (%) |
|---|---|---|---|
| (Dec. 24, 2004) | 0 | 95 | 94 |
| Jan. 25, 2005 | 1-Month | 95 | 93 |
| Mar. 16, 2005 | 3-Month | 95 | 93 |
| Jul. 12, 2005 | 6-Month | 95 | 95 |
| Mar. 27, 2006 | 15-Month | 93 | 99 |
| Jan. 17, 2007 | >2 Years | 95 | — |

Coupling of Tandem DNA Oligonucleotides from the Phosphoramidite Linker

This experiment was carried out to show proof of concept that the inventive phosphoramidite linkers cleaved from the 3′ region of the upstream DNA strand and the 5′ region of the downstream DNA strand of both oligonucleotides in tandem. Analysis was carried out using reverse-phase high performance liquid chromatography (HPLC).

(SEQ ID NO:1)

5′-TTTTTTTTTTTTTTTTTTTT_Linker-T_TTTTTTTTTT-3′

### Poly T Oligomers Adjoined with T-Succinyl Phosphoramidite Linker

Starting from the 3′ end, the downstream Poly T 10 mer was synthesized DMT-ON using a 0.2 μmol scale. Afterwards, the T succinate phosphoramidite linker was added using a 1 μmol scale synthesis cycle.

The overall coupling efficiency (CE) of the upstream oligo was ˜99.3 percent. After linker addition, the CE dropped to 79 percent. During its detritylation a light orange color was observed within the synthesis column suggesting the linker had been coupled to the 5′ region of the downstream oligo. The final CE of the two oligos in tandem was ˜85 percent.

Cleavage and Deprotection of Tandem Oligos from the Phosphoramidite Linker

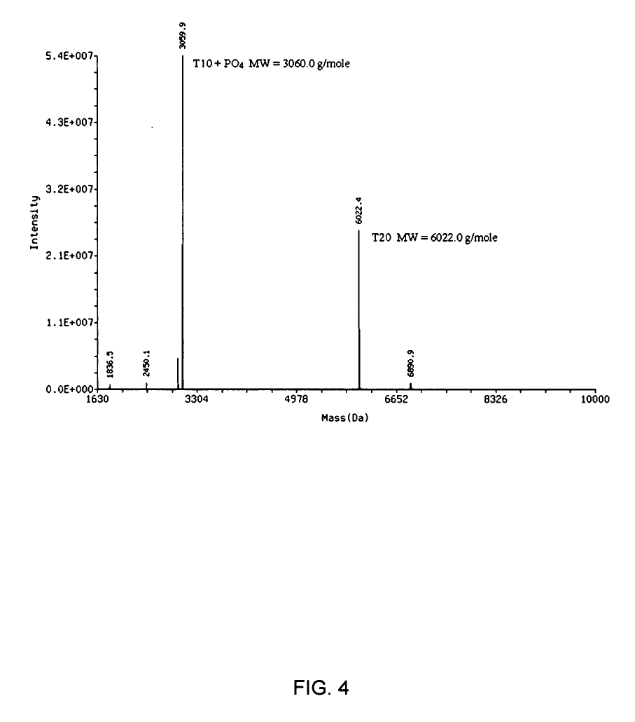

The coupled oligonucleotides were cleaved from their support using 28-30% ammonium hydroxide in solution (NH₄OH). One mL NH₄OH was passed through each sample 3 times with a hold time of fifteen minutes using Norm-Ject 1 mL syringes (4010.200V0). Following cleavage from the solid support, the tandem T₁₀ and T₂₀ oligos were deprotected over night (O/N) at 55° C. Though the thymidine phosphoramidite has no base protection, exposure to NH₄OH will remove any residual cyanoethyl groups from the oligonucleotide. All samples were normalized to 100 μM in water. FIG. 4 shows LC-MS data for T₃₀ (SEQ ID NO:1) cleaved into T₁₀ and T₂₀ oligos.

Biological Functionality of Cleaved and Deprotected Tandem Oligos in PCR Reactions

pUC19 primers (56.3/55.8 Tₘs, respectively) were synthesized in tandem as described above.

| | |
|---|---|
| 5′-GATACGGGAGGGCTTACCA(linker-T)GATAACACTGCGGCCAACTT-3′ | (SEQ ID NO:2) |

When cleaved and deprotected, as described above, the resulting primers are:

(SEQ ID NO:3)

(Forward) 5′-GATACGGGAGGGCTTACCAT-3′

(SEQ ID NO:4)

(Reverse) 5′-PO4-GATAACACTGCGGCCAACTT-3′

PCR was carried out on a GeneAmp PCR System 9700 (Applied Biosystems) using the following PCR cycle:
1. 94° C., 10 min
2. 94° C., 0:30 sec
3. 55° C., 0:45 sec
4. 73° C., 2:00 min
5. Repeat steps 2-4, 30 ×
6. 72° C., 7 min
7. 4° C., ∞

Reagents purchased from Applied Biosystems included PCR Buffer II 10×, 25 mM MgCl₂, 125 mM dNTPs, 3.2 pmol forward and reverse primers, and 5 U AmpliTaq Gold enzyme.

After PCR, the samples were analyzed on a 0.9% Agarose gel with EtBr. Fermentas O'Generuler 50 pb DNA Ladder (0.1 μg/μL) was used to measure amplicon size.

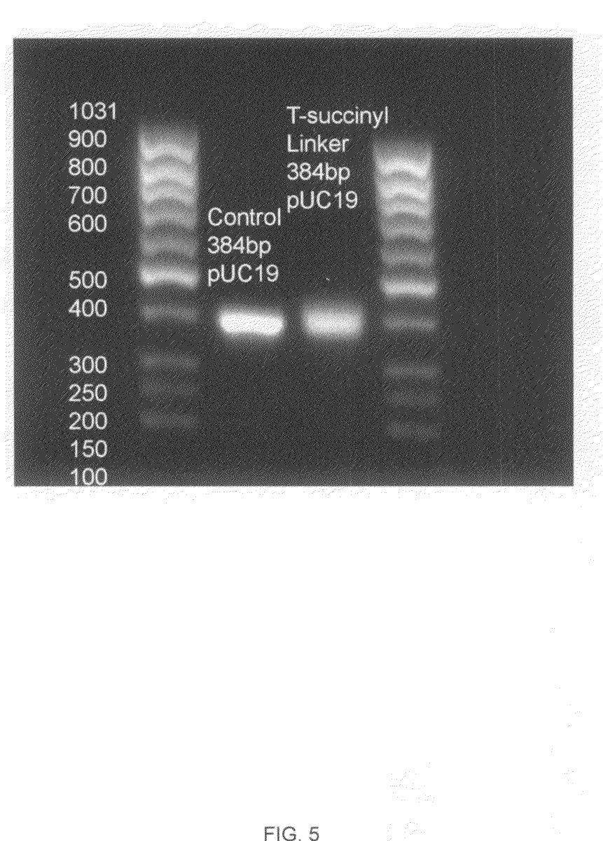

FIG. 5 shows a gel image comparing control primers and NH₄OH(l) cleaved T-succinyl Linker primers in Tandem. Based on data obtained from MS and HPLC, the cleavage between upstream and downstream poly T oligonucleotides is not 50:50 (see FIG. 4). For the application of PCR, having more of one primer than the other in solution could have an effect on the amplification. The target [c] for each of the control primers is 3.2 pmol. To calculate the initial [c] of the primers in tandem, an average of both extinction coefficients was taken. The final [c] value apparently was an overestimation, hence the lighter band intensity of the custom linker sample compared with the control.

Synthesis of a DNA Oligonucleotide Using the Inventive Phosphoramidite Linker Material

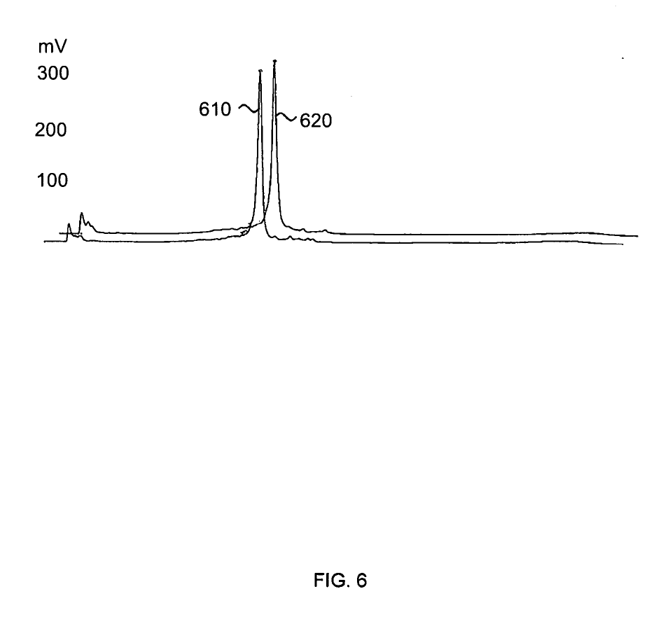

As shown in FIG. 6, the utility of a succinate (tandem) linker phosphoramidite as a universal support was tested by synthesizing a thymidine 10 mer homopolymer (T₁₀) (SEQ ID NO:5) on a standard polystyrene support (610) and on bare glass (620). Use of the phosphoramidite in this manner shows that high quality oligonucleotides may be synthesized directly from a glass surface, such as the SiO₂ layer of a silicon chip or microarray, or on underivitized glass supports. The quality of the two oligonucleotides, one synthesized on a standard support and the other synthesized directly on bare glass, are virtually indistinguishable, suggesting further that this linker is suitable for microarray work.

The two oligonucleotides were synthesized on an Applied Biosystems 394 synthesizer. The control oligo (610) was produced using a polystyrene flow-through column with the first nucleoside (thymidine) attached to the support. The control oligo, after synthesis was completed, was removed from the solid support by treatment in 28% ammonium hydroxide solution, a standard cleavage reagent. The oligo was heated to 55° C. for 30 min to remove cyanoethyl groups from the oligomer. The same T₁₀ homopolymer was also synthesized on an aminated glass support from CPG, Inc., using the same synthesizer cycle as the control oligo with two exceptions: 1) since the first oligonucleotide is not pre-attached to the bare support, the tandem oligo linker was coupled to the support in the same manner as all other bases were coupled (BTT activator plus amidite) except the first coupling step was performed for 15 minutes instead of 30 seconds, which is adequate for subsequent additions. The need for the longer coupling time is two-fold: the tandem oligo linker has a higher molecular weight than standard phosphoramidites and therefore is expected to react more slowly than standard phosphoramidites, and 2) the primary amine on the CPG support is less reactive than the hydroxyl that is normally present on a standard RNA/DNA synthesis support. Subsequent nucleosides were attached in the same manner for each oligonucleotide.

This proof-of-concept reaction proves that DNA may also be synthesized in situ directly on a DNA chip (microarray). The utility of this concept is straightforward, because it will allow synthesis of oligonucleotides on a highly parallel microarray platform, and allows the oligonucleotides to be removed from the microarray for use in assays. The most obvious applications for collecting oligonucleotides from a microarray synthesis are: 1) synthesis of synthetic genes from the oligos, and 2) use of the oligos in large pools for genotyping applications such as MIP (molecular inversion probe) genotyping.

Synthesis and Cleavage of Tandem RNA Oligonucleotides Using the Inventive Phosphoramidite Linker

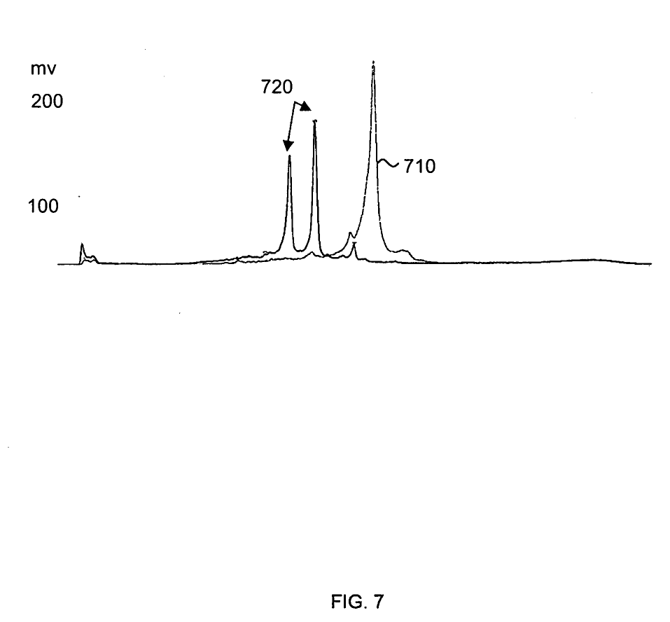

The utility of the inventive phosphoramidite linker material was also tested for use in RNA synthesis. Although RNA and DNA phosphoramidites each have the same reactive groups to facilitate coupling reactions, proof of the ability to synthesis two RNA fragments in a single reaction vessel is desirable. With a clear application in siRNA research, the most common application is where two complementary ssRNA fragments are pooled and hybridized to form an siRNA cassette. Synthesis of two RNA fragments in a single reaction vessel so that errors associated with incorrect pooling of the two ssRNA strands is avoided. The chromatogram shown in FIG. 7 is of a 40 mer deoxyuridine (dU) with two thymidine (dT) nucleosides in the sequence (SEQ ID NO:6), linked in tandem and then cleaved into two shorter RNA fragments. A control ribooligomer (SEQ ID NO:7) is 40 nt in length but lacks the internal succinate linker amidite, and thus should not cleave under treatment with alkali solution. After treatment of both RNA oligos with 28% ammonium hydroxide, the 40 mer synthesized with an internal tandem oligo linker fragments into two smaller RNA oligonucleotides (720). The control 40 nt ribooligomer that was synthesized without a tandem linker did not cleave when treated with ammonium hydroxide. The fragments were both analyzed on RP-HPLC to show that the uncleaved control fragment migrates slower on the column (710) relative to the shorter cleaved fragments.

As one of ordinary skill in the art will appreciate, various changes, substitutions, and alterations could be made or otherwise implemented without departing from the principles of the present invention. Accordingly, the scope of the invention should be determined by the following claims and their legal equivalents.

What is claimed is:

1. A method of synthesizing a compound having formula I
wherein: 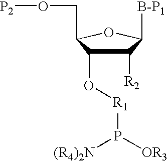
B is a nucleobase;
P₁ is an acyl, an aroyl, a phenoxyacetyl, an isopropylphenoxyacetyl, a t-butylphenoxyacetyl, an acetyl, a benzoyl, an isobutyryl, a levulinoyl, a dialkylformamidino, an fmoc, or a photolabile protecting group;
P₂ is a dimethoxytrityl, a monomethoxytrityl, a levulinoyl, an ArCO, a silyl, or a photolabile protecting group;
R₁ is a base-labile group;
R₂ is a hydrogen, a fluoro, a protected amino, a protected hydroxyl, an O-alkyl, an O-alkylalkoxy, or a secondary amine;
R₃ is a phosphorus protecting group;
R₄ is an alkyl or (R₄)₂ forms a cyclic secondary amine; and
O, P, and N have their normal meanings of oxygen, phosphorous and nitrogen.
comprising:
a) providing a compound with formula III 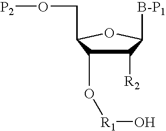
wherein:
B is a nucleobase;
P₁ is an acyl, an aroyl, a phenoxyacetyl, an isopropylphenoxyacetyl, a t-butylphenoxyacetyl, an acetyl, a benzoyl, an isobutyryl, a levulinoyl, a dialkylformamidino, an fmoc, or a photolabile protecting group;
P₂ is a dimethoxytrityl, a monomethoxytrityl, a levulinoyl, an ArCO, a silyl, or a photolabile protecting group;
R₁ is a base-labile group;
R₂ is a hydrogen, a fluoro, a protected amino, a protected hydroxyl, an O-alkyl, an O-alkylalkoxy, a secondary amine; and
O and H have their normal meanings of oxygen and hydrogen;
b) reacting said compound having formula III with 1-1.5 equivalents of an O-protected bis-dialkylaminophosphodiamidite, (R′₁O—P—(NR′₂)₂ wherein R₂ is a dialkyl or (NR′₂)₂ forms a cyclic secondary amine and R′₁ is a protecting group; or
c) reacting said compound having formula III with 1-1.5 equivalents of chloro-β-cyanoethyl-N′N′-diisopropylphosphoramidite in the presence of a tertiary amine.

2. The method as set forth in claim 1, wherein R′₂ is a diisopropyl, or (NR′₂)₂ forms a piperidine, or a morpholine.

3. The method as set forth in claim 1, wherein R′₁ is a methyl, a β-cyanoethyl, an allyl, or a nitrophenethyl.

4. The method as set forth in claim 1, wherein said O-protected bis-dialkylaminophosphodiamidite is β-cyanoethyl-N,N,N′,N′,-tetraisopropylphosphordiamidite.

5. The method as set forth in claim 1, further comprising adding 0.05-1.5 equivalents of an activator to said O-protected bis-dialkylaminophosphodiamidite.

6. The method as set forth in claim 5, wherein said activator is 1H-tetrazole, S-ethylthiotetrazole, 5-benzylthio-1H-tetrazole, 4,5-dicyanoimidiazole, a trifluoromethylsulfonic acid salt, or a pyridinium salt.

7. The method as set forth in claim 1, wherein said reacting is accomplished at room temperature.

8. The method as set forth in claim 1, wherein said reacting is accomplished for between about 1 and about 5 hours.

9. The method as set forth in claim 1, wherein R₁ is 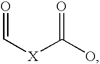
wherein x is an alkyl, an alkoxyalkyl, an aryl aralkyl, or an ether.

10. The method as set forth in claim 1, wherein R₁ is a succinate, a malonate, a glutarate, an adipate, a diglycolate, a catechol, or an analog or derivative thereof.

11. The method as set forth in claim 1, further comprising adding dichloromethane when said reacting is complete to form a solution.

12. The method as set forth in claim 11, further comprising washing said solution with a 5% aqueous NaHCO₃ solution and a saturated NaCl solution.

13. The method as set forth in claim 11, further comprising isolating an organic phase of said solution, wherein said organic phase contains said compound having formula 1.

14. The method as set forth in claim 13, further comprising drying said organic phase with Na₂SO₄.

15. The method as set forth in claim 13, further comprising filtering said organic phase.

16. The method as set forth in claim 13, further comprising evaporating said organic phase, wherein said evaporating dries said organic phase.

17. The method as set forth in claim 13, wherein said isolating is accomplished at a pH in the range of between about 7.5 and about 9.5.

18. The method as set forth in claim 1, wherein said compound having formula I is stable for at least 2.5 years at −40° C.

19. The method as set forth in claim 1, wherein said compound having formula I is stable for at least two days at ambient temperature.

20. The method as set forth in claim 1, further comprising coupling said compound having formula I to a solid support.

21. The method as set forth in claim 20, wherein said solid support is a solid support matrix, a controlled pore glass, a polystyrene, or an oligonucleotide array.


SEQUENCE LISTING (7 sequences)

SEQ ID NO: 1 - DNA, 30 bp, Artificial Sequence
  Description of Artificial Sequence Synthetic oligonucleotide
tttttttttttttttttttntttttttttt

SEQ ID NO: 2 - DNA, 40 bp, Artificial Sequence
  Description of Artificial Sequence Synthetic oligonucleotide
gatacgggagggcttaccangataacactgcggccaactt

SEQ ID NO: 3 - DNA, 20 bp, Artificial Sequence
  Description of Artificial Sequence Synthetic oligonucleotide
gatacgggagggcttaccat

SEQ ID NO: 4 - DNA, 20 bp, Artificial Sequence
  Description of Artificial Sequence Synthetic oligonucleotide
gataacactgcggccaactt

SEQ ID NO: 5 - DNA, 10 bp, Artificial Sequence
  Description of Artificial Sequence Synthetic oligonucleotide
tttttttttt

SEQ ID NO: 6 - DNA, 40 bp, Artificial Sequence
  Description of Artificial Sequence Synthetic oligonucleotide
uuuuuuuuuuuuuuuuuuutnuuuuuuuuuuuuuuuuuuu

SEQ ID NO: 7 - RNA, 40 nt, Artificial Sequence
  Description of Artificial Sequence Synthetic oligonucleotide
uuuuuuuuuuuuuuuuuuuuuuuuuuuuuuuuuuuuuuuu